(19) United States
(12) Patent Application Publication
(10) Pub. No.: US 2018/0256132 A1
(43) Pub. Date: Sep. 13, 2018

(54) METHODS AND SYSTEMS FOR OPERATING AN ULTRASOUND SYSTEM ACCORDING TO A PROTOCOL

(71) Applicant: General Electric Company, Schenectady, NY (US)

(72) Inventors: Menachem Halmann, Wauwatosa, WI (US); Mitsuhiro Nozaki, Nerima-ku (JP)

(21) Appl. No.: 15/454,001

(22) Filed: Mar. 9, 2017

(51) Int. Cl. A61B 8/00; A61B 8/08

(52) U.S. Cl. CPC A61B 8/40; A61B 8/4227; A61B 8/4254; A61B 8/488; A61B 8/4236

(57) ABSTRACT

A method and system are provided for operating an ultrasound system. The method and system acquire ultrasound data with an ultrasound probe according to at least one of: i) an examination protocol while maintaining an ultrasound probe against a patient region of interest as a patient is moved between first and second examination related positions in connection with the examination protocol; or ii) a monitoring protocol during which an ultrasound probe is maintained against a patient region of interest. The method and system collect probe position information and based on the probe position information, identify a patient movement based (PMB) transition of the probe. The method and system direct the ultrasound system to automatically perform an examination related task in connection with identifying the PMB transition.

### FIELD

Embodiments described herein generally relate to operating an ultrasound system according to a protocol while maintaining an ultrasound probe against a patient region of interest.

### BACKGROUND OF THE INVENTION

Ultrasound systems are utilized to analyze patients in a variety of manners based on various examination procedures. Some examination procedures call for the collection of ultrasound images and data while the patient is oriented in different positions. One limitation of conventional ultrasound systems is that, in connection with certain examination procedures, more than one individual or ultrasound operator must be present to perform the examination procedure. For example, the examination procedure for a “Passive Leg Raise” (PLR) procedure calls for the patient to be moved between different positions, with separate ultrasound data sets acquired while the patient is in each requisite position. Conventionally, one individual was necessary to assist and guide the patient between the appropriate positions, while another individual operated the ultrasound system and held the ultrasound probe on the patient's skin. Accordingly, multiple individuals were required to be present with the patient when performing a PLR examination protocol, as well as numerous other types of examination protocols.

Further, conventional ultrasound systems experience certain limitations in connection with modes of operation that utilize gating functions. In certain modes of operation, the ultrasound system defines a gate within the region of interest, for which ultrasound data is acquired. Doppler ultrasound information is collected for the region within the gate and utilized in connection with various Doppler related functions. However, when a patient moves, the position of the ultrasound probe shifts. Additionally or alternatively, when a patient experiences movement, the location and/or shape of anatomy changes within the region of interest. Changes in the probe position and/or changes in the location/shape of anatomy may cause characteristics of interest to move outside of the Doppler gate, which adversely affects the functionality of the ultrasound system in connection with obtaining Doppler related ultrasound data.

### BRIEF DESCRIPTION OF THE INVENTION

In one embodiment, a method is provided for operating an ultrasound system, the method comprising acquiring ultrasound data with the ultrasound probe according to at least one of: i) an examination protocol while maintaining an ultrasound probe against a patient region of interest as a patient is moved between first and second examination related positions in connection with the examination protocol; or ii) a monitoring protocol during which an ultrasound probe is maintained against a patient region of interest. The method collects probe position information and based on the probe position information, identifies a patient movement based (PMB) transition of the probe. The method directs the ultrasound system to automatically perform an examination related task in connection with identifying the PMB transition.

Optionally, the ultrasound data is acquired in connection with an examination protocol and the PMB transition is identified in connection with the first and second examination related positions, the examination related task includes automatically labeling a portion of the ultrasound data with a label related to the second examination related position. Optionally, the method further comprises forming images from the ultrasound data, wherein the examination related task includes automatically labeling a select image from the images with a label indicating that the select image was acquired when the patient was in the second examination related position. Optionally, ultrasound data is acquired in connection with an examination protocol and the PMB transition is identified in connection with the first and second examination related positions, the ultrasound data comprises first and second ultrasound data sets that are acquired in connection with the first and second examination related positions, respectively, the examination related task including automatically labeling the second ultrasound data set with a label indicating whether the second ultrasound data set was acquired while the patient was in the first or second examination related positions or another examination related position.

Optionally, the examination related task includes automatically triggering acquisition and storage of the ultrasound data in connection with one or more PMB transitions during at least one of the examination or monitoring protocols. Optionally, the examination related task includes automatically adjusting a position of a Doppler gate used in connection with acquiring at least a portion of the ultrasound data. Optionally, the ultrasound data is acquired in connection with an examination protocol and the PMB transition is identified in connection with the first and second examination related positions, the examination related task includes automatically adjusting one or more i) an acquisition parameter or ii) an imaging mode, related to acquiring the ultrasound data, in connection with the second examination related position.

Optionally, the method further comprises forming images from the ultrasound data, wherein the examination related task includes automatically labeling a select image from the images with a label indicating that the select image was acquired when the patient was in an examination related position. Optionally, the probe position information is collected from at least one of a position sensor provided with the probe and/or an analysis of the ultrasound data. Optionally, the identifying operation comprises comparing a position limit to the probe position information as the probe position information is updated to indicate a current position of the probe as the patient is moved from a first examination related position to a second examination related position, the position limit defined relative to a reference position corresponding to a probe position when the patient is in the first examination related position.

In an embodiment, an ultrasound system is provided comprising of an ultrasound probe configured to be maintained against a patient proximate a region of interest. The system includes an ultrasound probe configured to be maintained against a patient proximate a region of interest, the ultrasound probe configured to acquire ultrasound data during at least one of: i) an examination protocol while maintaining an ultrasound probe against a patient region of interest as a patient is moved between first and second examination related positions in connection with the examination protocol; or ii) a monitoring protocol during which an ultrasound probe is maintained against a patient region of interest. The system includes a position sensor provided with the ultrasound probe, the ultrasound sensor to collect probe position information. The system includes memory to store the ultrasound data and to store instructions, and a processor that, when executing the instructions, is configured to: identify a patient motion based (PMB) transition of the patient in connection with the first and second examination related positions based on the probe position information; and direct the ultrasound system to automatically perform an examination related task in connection with identifying the PMB transition.

Optionally, the system further includes a probe mounting element to secure the probe to the patient. Optionally, the ultrasound data is acquired in connection with an examination protocol and the PMB transition is identified in connection with the first and second examination related positions, wherein the examination related task includes automatically labeling a portion of the ultrasound data with a label indicating whether the select image was acquired when the patient was in the first or second examination related positions, or another examination related position.

Optionally, the memory stores ultrasound data comprises first and second ultrasound data sets that are acquired in connection with first and second examination related positions, respectively, the examination related task including automatically labeling the second ultrasound data set with a label indicating that the second ultrasound data set was acquired while the patient was in the second examination related position. Optionally, the processor, when executing the instructions, is configured to form images from the ultrasound data, wherein the examination related task includes automatically labeling a select image from the images with a label indicating that the select image was acquired when the patient was in an examination related position. Optionally, the processor is configured to automatically trigger, as the examination related task, acquisition and storage of the ultrasound data in connection with one or more PMB transitions during at least one of the examination or monitoring protocols (e.g., a second ultrasound data set related to a second examination related position).

Optionally, the processor is configured to automatically adjust, as the examination related task, a position of a Doppler gate used in connection with acquiring at least a portion of the ultrasound data. Optionally, the ultrasound data is acquired in connection with an examination protocol and the PMB transition is identified in connection with the first and second examination related positions, wherein the processor is further configured to form images from the ultrasound data and to automatically label a select image from the images with a label indicating that the select image was acquired when the patient was in the second examination related position.

In one embodiment, an ultrasound system is provided, comprised of an ultrasound probe configured to acquire ultrasound data. The system includes a probe mounting element to secure the probe at a tissue attachment location on a patient and a position sensor provided with the ultrasound probe. The ultrasound sensor collects probe position information. Memory stores the ultrasound data and instructions. The system includes a processor that, when executing the instructions, is configured to: identify a patient motion based (PMB) transition of the probe with respect to the tissue attachment location and direct the ultrasound system to automatically perform an examination related task in connection with identifying the PMB transition.

Optionally, the processor is further configured to detect non-physician directed motion of the probe relative to the tissue attachment location. Optionally, the processor is further configured to automatically adjust, as the examination related task, a position of a Doppler gate used in connection with acquiring at least a portion of the ultrasound data in response to detection of the non-physician directed motion.

### BRIEF DESCRIPTION OF THE DRAWINGS

FIG. 1 is a schematic diagram of an ultrasound imaging system formed in accordance with an embodiment herein.

FIG. 2 illustrates an ultrasound probe, configured as a patch probe, for use in accordance with embodiments herein.

FIG. 3 illustrates examples of positions, in which a patient may be positioned during a PLR examination protocol.

FIG. 4 illustrates the operations of a PLR examination protocol implemented in accordance with embodiments herein.

FIG. 5 illustrates a process carried out in accordance with embodiments herein for operating an ultrasound system according to an examination protocol.

FIG. 6 illustrates a process carried out in accordance with embodiments herein.

FIG. 7 illustrates a process carried out in accordance with embodiments herein.

### DETAILED DESCRIPTION OF THE INVENTION

The following detailed description of certain embodiments will be better understood when read in conjunction with the appended drawings. To the extent that the FIGS. illustrate diagrams of the functional modules of various embodiments, the functional blocks are not necessarily indicative of the division between hardware circuitry. Thus, for example, one or more of the functional blocks (e.g., processors or memories) may be implemented in a single piece of hardware (e.g., a general purpose signal processor or a block of random access memory, hard disk, or the like). Similarly, the programs may be stand-alone programs, may be incorporated as subroutines in an operating system, may be functions in an installed software package, and the like. It should be understood that the various embodiments are not limited to the arrangements and instrumentality shown in the drawings.

As used herein, an element or step recited in the singular and proceeded with the word “a” or “an” should be understood as not excluding plural of said elements or steps, unless such exclusion is explicitly positioned. Furthermore, references to “one embodiment” of the present invention are not intended to be interpreted as excluding the existence of additional embodiments that also incorporate the recited features. Moreover, unless explicitly positioned to the contrary, embodiments “comprising” or “having” an element or a plurality of elements having a particular property may include additional elements not having that property.

The terms “examination protocol” and “examination procedure” are used interchangeably to refer to standardized procedures, physician specific procedures and patient specific procedures, in which ultrasound data is acquired during at least first and second acquisition intervals that are spaced apart by at least a brief period of time. Between the first and second acquisition intervals, some aspect of the patient's anatomy is physically moved or adjusted, such as changing and orientation the patient's body, raising or lowering one or more limbs, undergoing physical exertion (e.g., a stress test) and the like. The examination protocol/procedure includes standardized procedures adopted by multiple positions, as well as individualized procedures that may be utilized by only a single physician or a handful of physicians.

The term “monitoring protocol” refers to long-term or short-term continuous or intermittent monitoring of a patient, during which a physician, clinician or ultrasound operator may not be present in a room. During a monitoring protocol, healthcare personnel may periodically visit and check up on the patient, but do not necessarily directly operate the ultrasound system. For example, after a procedure, a patient may be located in a hospital room, recovery room, or post-operative room. While the patient is in recovery, in accordance with embodiments herein, ultrasound systems monitor one or more characteristics of the patient, such as heart rate, blood pressure, stroke volume and the like. Additionally or alternatively, ultrasound systems may be utilized to monitor non-cardiac physiologic conditions of the patient, such as to monitor the lungs, internal organs and the like.

The term “physician” is used to generally refer to doctors, nurses, clinicians, ultrasound operators and other medical personnel.

The terms “patient motion based transition” or “PMB transition” are used in connection with movement of a patient and/or movement of a probe. For example, a patient may transition between various positions in connection with an examination protocol (e.g., moving the patient between raised and lowered positions during a passive leg raise examination protocol) or a monitoring protocol. A probe may transition between different positions and/or orientations. The term transition, when referring to movement of the probe, refers to non-physician directed movement of the probe. A non-limiting example of non-physician directed movement includes patient re-orientation movement, in which a patient is repositioned or reoriented, in accordance with an examination procedure or while being monitored. When the patient is being reoriented or repositioned, the probe remains at a fixed position on the patient, but moves relative to the room or earth, as the patient is moved. Additionally or alternatively, non-physician directed movement includes when the probe “unintentionally” moves relative to the patient while the patient remains stationary. The term transition, when referring to movement of the probe, does not include physician directed movement. Physician directed movement refers to movement of a probe relative to a patient's tissue, such as when a physician moves (e.g., sweeps, tilts, rotates) a hand-held probe relative to tissue to obtain ultrasound images from different positions and orientations.

For example, a patch type probe may be affixed to a patient for long-term monitoring before, after or otherwise separate from a physician being present and performing a specific examination procedure. Over time the patch may move relative to the patient (e.g., a mounting strap may shift, adhesive may partially or fully release a tissue-patch bond). Another example of transition is when a patch type probe remains affixed to patient tissue, but the patient moves or changes position (e.g., rolls over in a hospital bed, sits up, stands, bends over, etc.). The transition may refer to a transition point or position range, at which the patient is no longer in the first examination related position. The transition may represent a transition point or position range, in which the patient achieves the second examination related position. The transition may identify when the patient is in an intermediate position between the first and second examination related positions.

As explained herein, the ultrasound data may be acquired only when the patient is in select positions and/or acquired at select points in time (e.g., periodically). Optionally, ultrasound data may be acquired while the patient is moved from the first examination related position toward the second examination related position, through all or a portion of an intermediate position between the first and second examination related positions.

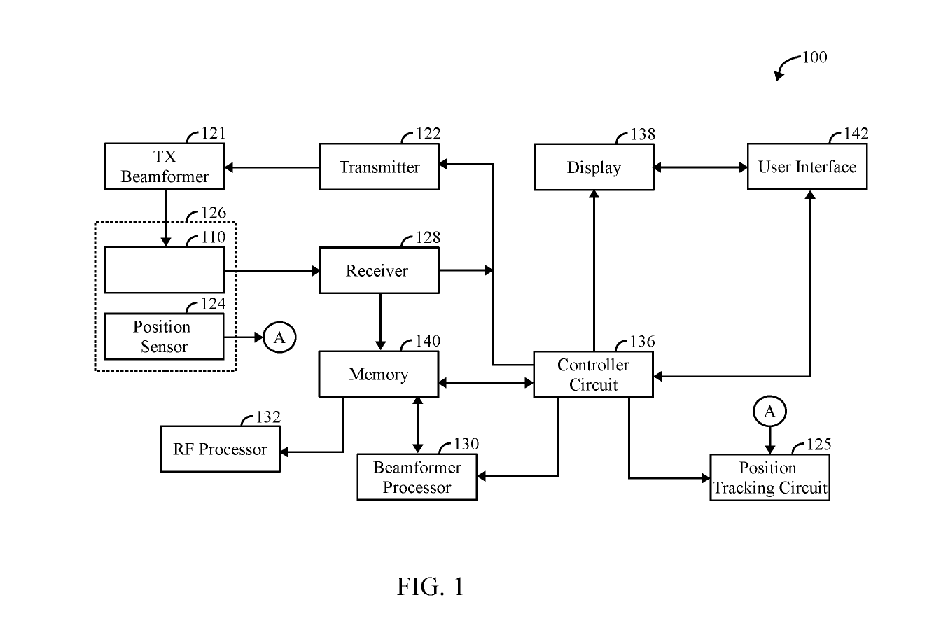

FIG. 1 is a schematic diagram of an ultrasound imaging system 100 formed in accordance with an embodiment herein. The ultrasound imaging system 100 includes an ultrasound probe 126 having a transducer 110 with one or more transducer elements. The probe 126 may represent various types of hand-held probes that are held in manipulated by a operator during operation. Additionally or alternatively, the probe 126 may be secured to the patient's skin throughout an examination or monitoring protocol without being held by an operator. As one example, the probe 126 may represent a patch type probe having a securing member, such as adhesive, Velcro strap and the like, to securely maintain the transducer 110 against the surface of the patient proximate to a region of interest.

As non-limiting examples, embodiments herein may utilize one or more of the ultrasound probes described in the following patents and patent applications: U.S. Pat. No. 7,125,383, titled “Method and apparatus for ultrasonic continuous, non-invasive blood pressure monitoring”, U.S. Pat. No. 7,425,199, titled “Method and apparatus for ultrasonic continuous, non-invasive blood pressure monitoring”, U.S. Pat. No. 7,621,876, titled “Continuous, Non-Invasive Technique for Determining Blood Pressure Using a Transmission Line Model and Transcutaneous”, U.S. Pat. No. 8,388,544, titled “Continuous, Non-Invasive Technique for Determining Blood Pressure Using a Transmission Line Model and Transcutaneous”, U.S. Pat. No. 9,204,857, titled “System and Method for Monitoring Hemodynamic State”, U.S. Pat. No. 8,414,495, titled “Ultrasound Patch Probe with Micro-Motor”, US Application 2015-0182187, titled “System and Method for Tracking an Invasive Device Using Ultrasound Position Signals”, and U.S. Pat. No. 7,470,232, titled “Method and Apparatus for Non-Invasive Ultrasonic Fetal Heart Rate Monitoring”, the complete subject matter of which are all expressly incorporated by reference in their entireties.

The probe 126 also includes a position sensor 124 obtain probe position information utilizing various techniques and provide the probe position information to the imaging system 100. By way of example, the position sensor 124 may represent a gyroscope or accelerometer that detects relative change in the position and/or orientation of position sensor 124. The change in the position and/or orientation is provided as probe position information to a position tracking circuit 125. The probe position information may be supplied through a wireless communications link, through a physical cable or otherwise. The probe position information may be conveyed along the same or a different communications link or physical cable as the ultrasound data. The position tracking circuit 125 may be provided integral within the imaging system 100 or as a separate stand-alone device that communicates with the imaging system 100. While the position tracking circuit 125 is illustrated as a separate block from the control circuit 136, it is understood that the position tracking circuit 125 may be implemented by one or more common or different processors as the processor 136. The probe position information is used to identify PMB transition.

Additionally or alternatively, the position sensor 124 and position tracking circuit 125 may include a combination of transmitters/receivers that communicate with one another to determine an absolute position and orientation of the position sensor 124 relative to a reference coordinate system. For example, the position tracking circuit 125 may include transmitters/receivers positioned on the ultrasound system 100, on the patient and/or elsewhere in the examination room, where the transmitters/receivers communicate with the position sensor 124 electrically, optically, magnetically, through radiofrequency signals and the like.

The ultrasound probe 126 is configured to be maintained against a patient proximate a region of interest. Additionally or alternatively, the ultrasound probe 126 is configured to acquire ultrasound data during an examination protocol while maintaining an ultrasound probe against a patient region of interest as a patient is moved between first and second examination related positions in connection with the examination monitoring protocol during which an ultrasound probe is maintained against a patient region of interest. The ultrasound probe 126 is configured to acquire ultrasound data or information from a region of interest (ROI) (e.g., organ, blood vessel, heart, brain, fetal tissue, cardiovascular, neonatal brain, embryo, abdomen, and/or the like) that includes one or more anatomical structures of the patient. The ultrasound data is acquired in connection with an examination protocol and the PMB transition is identified in connection with the first and second examination related positions, wherein the examination related task includes automatically labeling a portion of the ultrasound data with a label related to the second examination related position. The ultrasound data is acquired in connection with an examination protocol and the PMB transition is identified in connection with the first and second examination related positions, wherein the processor is further configured to form images from the ultrasound data and to automatically label a select image from the images with a label indicating whether the select image was acquired when the patient was in the first or second examination related positions, or another examination related position.

The ultrasound probe 126 is communicatively coupled to the processor 136 via the transmitter 122 and receiver 128. The transmitter 122 transmits a signal to a transmit beamformer 121 based on acquisition parameters received by the processor 136. The acquisition parameters may define an amplitude, pulse width, frequency, and/or the like of the ultrasonic pulses emitted by the transducer elements 124. The transducer elements 124 emit pulsed ultrasonic signals into a patient (e.g., a body). The acquisition parameters may be adjusted manually or automatically by selecting a gain setting, power, time gain compensation (TGC), resolution, and/or the like from the user interface 142. Acquisition parameters may be adjusted, as described herein, based on PMB transition. The signal transmitted by the transmitter 122 in turn drives a plurality of transducer elements 124 within an array in the transducer 110. The transducer 110 may be a matrix array of transducer elements arranged to include an elevation direction and an azimuth direction. For example only, the transducer array may include an array of transducer elements along the azimuth plane and along the elevation plane to from a matrix array probe. Optionally, the transducer 110 may include a relatively simple transducer array (e.g., a 1-D array). In various embodiments, the array of transducer elements may not be equal along the azimuth and elevation planes. For example, the transducer array of the ultrasound probe may be arranged as a 1.5-D array, a 1.75-D array, and/or the like. Additionally or alternatively, the array of transducer elements may be equal along the azimuth and elevation planes.

The transducer 110 emits pulsed ultrasonic signals into a body (e.g., patient) or volume corresponding to the acquisition settings along one or more scan planes. The ultrasonic signals may include, for example, one or more reference pulses, one or more pushing pulses (e.g., shear-waves), and/or one or more pulsed wave Doppler pulses. At least a portion of the pulsed ultrasonic signals back-scatter from the ROI (e.g., coronary artery and/or the like) to produce echoes. The echoes are delayed in time and/or frequency according to a depth or movement, and are received by the transducer elements 124 within the transducer array. Ultrasonic data is received and signals may be used for imaging, for generating and/or tracking shear-waves, for measuring changes in position or velocity within the ROI (e.g., flow velocity, movement of blood cells), differences in compression displacement of the tissue (e.g., strain), and/or for therapy, among other uses. For example, the probe 126 may deliver low energy pulses during imaging and tracking, medium to high energy pulses to generate shear-waves, and high energy pulses during therapy.

The receiver 128 may include one or more amplifiers, an analog to digital converter (ADC), and/or the like. The receiver 128 may be configured to amplify the received echo signals after proper gain compensation and convert these received analog signals from each transducer element 124 to digitized signals sampled uniformly in time. The digitized signals representing the received echoes are stored on memory 140, temporarily. The digitized signals correspond to the backscattered waves received by each transducer element 124 at various times. After digitization, the signals still may preserve the amplitude, frequency, phase information of the backscatter waves.

The beamformer processor 130 may include one or more processors. Optionally, the beamformer processor 130 may include a central controller circuit (CPU), one or more microprocessors, or any other electronic component capable of processing inputted data according to specific logical instructions. Additionally or alternatively, the beamformer processor 130 may execute instructions stored on a tangible and non-transitory computer readable medium (e.g., the memory 140) for beamforming calculations using any suitable beamforming method such as adaptive beamforming, synthetic transmit focus, aberration correction, synthetic aperture, clutter reduction and/or adaptive noise control, and/or the like. Optionally, the beamformer processor 130 may be integrated with and/or apart of the processor 136. For example, the operations described being performed by the beamformer processor 130 may be configured to be performed by the processor 136.

The beamformer processor 130 performs beamforming on the digitized signals and outputs a radio frequency (RF) signal. The RF signal is then provided to an RF processor 132 that processes the RF signal. The RF processor 132 may generate different ultrasound image modes, such as B-mode, color Doppler (e.g., velocity, power, variance), tissue Doppler (e.g., velocity), Doppler energy, and/or the like for multiple scan planes or different scanning patterns. For example, the RF processor 132 may generate tissue Doppler data for multi-scan planes. The RF processor 132 gathers the information (e.g., I/Q, B-mode, color Doppler, tissue Doppler, and Doppler energy information) related to multiple data slices and stores the data information, which may include time stamp and orientation/rotation information, in the memory 140. The imaging mode may be adjusted based on PMB transition.

Alternatively, the RF processor 132 may include a complex demodulator (not shown) that demodulates the RF signal to form IQ data pairs representative of the echo signals. The RF or IQ signal data may then be provided directly to the memory 140 for storage (e.g., temporary storage). Optionally, the output of the beamformer processor 130 may be passed directly to the processor 136.

The processor 136 may be configured to process the acquired ultrasound data (e.g., RF signal data or IQ data pairs) and prepare frames of ultrasound image data for display on the display 138. The processor 136 may include one or more processors. Optionally, the processor 136 may include a central controller circuit (CPU), one or more microprocessors, a graphics controller circuit (GPU), or any other electronic component capable of processing inputted data according to specific logical instructions. Having the processor 136 that includes a GPU may be advantageous for computation-intensive operations, such as volume-rendering. Additionally or alternatively, the processor 136 may execute instructions stored on a tangible and non-transitory computer readable medium (e.g., the memory 140).

The processor 136 is configured to perform one or more processing operations according to a plurality of selectable ultrasound modalities on the acquired ultrasound data, adjust or define the ultrasonic pulses emitted from the transducer elements 124, adjust one or more image display settings of components (e.g., ultrasound images, interface components, positioning regions of interest) displayed on the display 138, and other operations as described herein. a processor that, when executing the instructions, is configured to: identify a patient motion based (PMB) transition of the patient in connection with the first and second examination related positions based on the probe position information; and direct the ultrasound system to automatically perform an examination related task in connection with identifying the PMB transition. The processor, when executing the instructions, is configured to form images from the ultrasound data, wherein the examination related task includes automatically labeling a select image from the images with a label indicating whether the select image was acquired when the patient was in the first or second examination related positions, or another examination related position. The processor is configured to automatically trigger, as the examination related task, acquisition and storage of the ultrasound data in connection with one or more PMB transitions during at least one of the examination or monitoring protocols (e.g., a second ultrasound data set related to a second examination related position). The processor is configured to automatically adjust, as the examination related task, a position of a Doppler gate used in connection with acquiring at least a portion of the ultrasound data. A processor that, when executing the instructions, is configured to: identify a patient motion based (PMB) transition of the probe with respect to the tissue attachment location; and direct the ultrasound system to automatically perform an examination related task in connection with identifying the PMB transition. The processor is further configured to detect non-physician directed motion of the probe relative to the tissue attachment location. The processor is further configured to automatically adjust, as the examination related task, a position of a Doppler gate used in connection with acquiring at least a portion of the ultrasound data in response to detection of the non-physician directed motion.

Acquired ultrasound data may be processed in real-time by the processor 136 during a scanning or therapy session as the echo signals are received. Additionally or alternatively, the ultrasound data may be stored temporarily in the memory 140 during a scanning session and processed in less than real-time in a live or off-line operation. Optionally, the processor 136 may be a collection of circuits and/or software modules, but may be implemented utilizing any combination of dedicated hardware boards, DSPs, one or more processors, FPGAs, ASICs, a tangible and non-transitory computer readable medium configured to direct one or more processors, and/or the like.

For example, the processor 136 may include circuits configured to process the IQ data pairs in a corresponding manner to generate, respectively, color flow data, ARFI data, B-mode data, spectral Doppler data, acoustic streaming data, tissue Doppler data, tracking data, electrography data (e.g., strain data, shear-wave data), among others, all of which may be stored in a memory 140 temporarily before subsequent processing. The data may be stored, for example, as sets of vector data values, where each set defines an individual ultrasound image frame. The vector data values are generally organized based on the polar coordinate system. The configured circuits may perform mid-processor operations representing one or more software features of the ultrasound imaging system 100. The processor 136 may receive ultrasound data in one of several forms. The received ultrasound data may constitute IQ data pairs representing the real and imaginary components associated with each data sample of the digitized signals. The IQ data pairs are provided to one or more of the circuits of the processor 136, for example, a color-flow circuit, an acoustic radiation force imaging (ARFI) circuit, a B-mode circuit, a spectral Doppler circuit, an acoustic streaming circuit, a tissue Doppler circuit, a tracking circuit, an electrography circuit, and/or the like. Other configured circuits may be included, such as an M-mode circuit, power Doppler circuit, among others. However, embodiments described herein are not limited to processing IQ data pairs. For example, processing may be done with RF data and/or using other methods. Furthermore, data may be processed through multiple circuits.

The memory 140 may be used for storing processed frames of acquired ultrasound data that are not scheduled to be displayed immediately or to store post-processed images (e.g., shear-wave images, strain images), firmware or software corresponding to, for example, a graphical user interface, one or more default image display settings, programmed instructions (e.g., for the processor 136, the beamformer processor 130, the RF processor 132), and/or the like. The memory 140 may be a tangible and non-transitory computer readable medium such as flash memory, RAM, ROM, EEPROM, and/or the like. The memory 140 may store ultrasound image data sets of the ultrasound data and related labels. The ultrasound data is acquired in connection with an examination protocol and the PMB transition is identified in connection with the first and second examination related positions, the examination related task includes automatically labeling a portion of the ultrasound data with a label related to the second examination related position. The memory may store images from the ultrasound data, wherein the examination related task includes automatically labeling a select image from the images with a label indicating that the select image was acquired when the patient was in the second examination related position. The ultrasound data is acquired in connection with an examination protocol and the PMB transition is identified in connection with the first and second examination related positions. The ultrasound data comprises first and second ultrasound data sets that are acquired in connection with the first and second examination related positions, respectively. The examination related task includes automatically labeling the second ultrasound data set with a label indicating that the second ultrasound data set was acquired while the patient was in the second examination related position. The examination related task includes automatically triggering acquisition and storage of the ultrasound data in connection with one or more PMB transitions during at least one of the examination or monitoring protocols (e.g., a second ultrasound data set). For example, a 3D ultrasound image data set may be mapped into the corresponding memory 140, as well as one or more reference planes. The processing of the ultrasound data, including the ultrasound image data sets, may be based in part on user inputs, for example, user selections received at the user interface 142. The memory stores ultrasound data comprising first and second ultrasound data sets that are acquired in connection with first and second examination related positions, respectively, the examination related task including automatically labeling the second ultrasound data set with a label indicating that the second ultrasound data set was acquired while the patient was in the second examination related position.

The processor 136 is operably coupled to a display 138 and a user interface 142. The display 138 may include one or more liquid crystal displays (e.g., light emitting diode (LED) backlight), organic light emitting diode (OLED) displays, plasma displays, CRT displays, and/or the like. The display 138 may display patient information, ultrasound images and/or videos, components of a display interface, one or more 2D, 3D, or 4D ultrasound image data sets from ultrasound data stored in the memory 140 or currently being acquired, measurements, diagnosis, treatment information, and/or the like received by the display 138 from the processor 136.

The user interface 142 controls operations of the processor 136 and is configured to receive inputs from the user. The user interface 142 may include a keyboard, a mouse, a touchpad, one or more physical buttons, and/or the like. Optionally, the display 138 may be a touch screen display, which includes at least a portion of the user interface 142. For example, a portion of the user interface 142 may correspond to a graphical user interface (GUI) generated by the processor 136 shown on the display. The GUI may include one or more interface components that may be selected, manipulated, and/or activated by the user operating the user interface 142 (e.g., touch screen, keyboard, mouse). The interface components may be presented in varying shapes and colors, such as a graphical or selectable icon, a slide bar, a cursor, and/or the like. Optionally, one or more interface components may include text or symbols, such as a drop-down menu, a toolbar, a menu bar, a title bar, a window (e.g., a pop-up window) and/or the like. Additionally or alternatively, one or more interface components may indicate areas within the GUI for entering or editing information (e.g., patient information, user information, diagnostic information), such as a text box, a text field, and/or the like.

In various embodiments, the interface components may perform various functions when selected, such as measurement functions, editing functions, database access/search functions, diagnostic functions, controlling acquisition settings, and/or system settings for the ultrasound imaging system 100 performed by the processor 136.

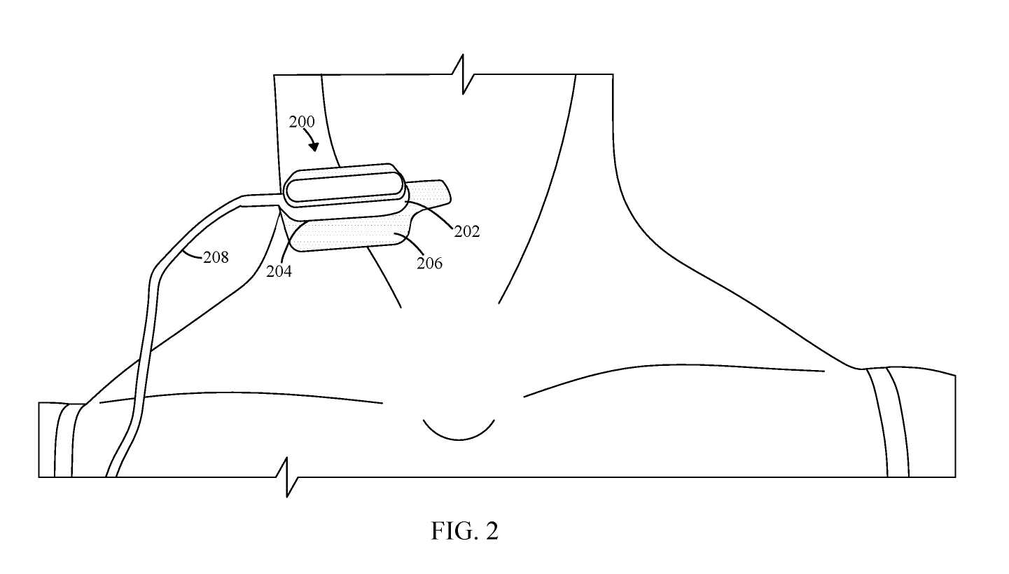

FIG. 2 illustrates an ultrasound probe, configured as a patch probe, for use in accordance with embodiments herein. The patch probe 200 is positioned at a desired location on a patient's skin, based upon an examination or monitoring protocol. In the example of FIG. 2, the patch probe 200 is located on a patient snacks proximate to the carotid artery. The patch probe 200 includes a housing 202 having a proximal surface 204 configured to directly engage tissue on the patient. It is recognized that an intervening layer of gel or other ultrasound matching material may be provided between the proximal surface 204 and the patient's skin. A probe mounting element 206 is formed with or secured to the housing 202. The probe mounting element 206 is configured to secure the probe 200 to a tissue attachment location on the patient. The probe mounting element 206 may be formed in alternative manners, based upon the type and nature of the probe 200, the anatomy to be analyzed and the nature of the examination protocol. For example, the probe mounting element 206 may be constructed as an adhesive layer having one portion secured to the housing 202, while another portion is secured to the tissue attachment location surrounding the region of interest to be imaged/analyzed. The patch probe 200 is coupled to a physical cable 208 that carries data and other information between the transducer (e.g. transducer 110 in FIG. 1) in the probe and the transmitter beamformer 121 and receiver 128 of the imaging system 100 (FIG. 1). The cable 208 may also carry probe position information to and from a position sensor (e.g., 124 in FIG. 1).

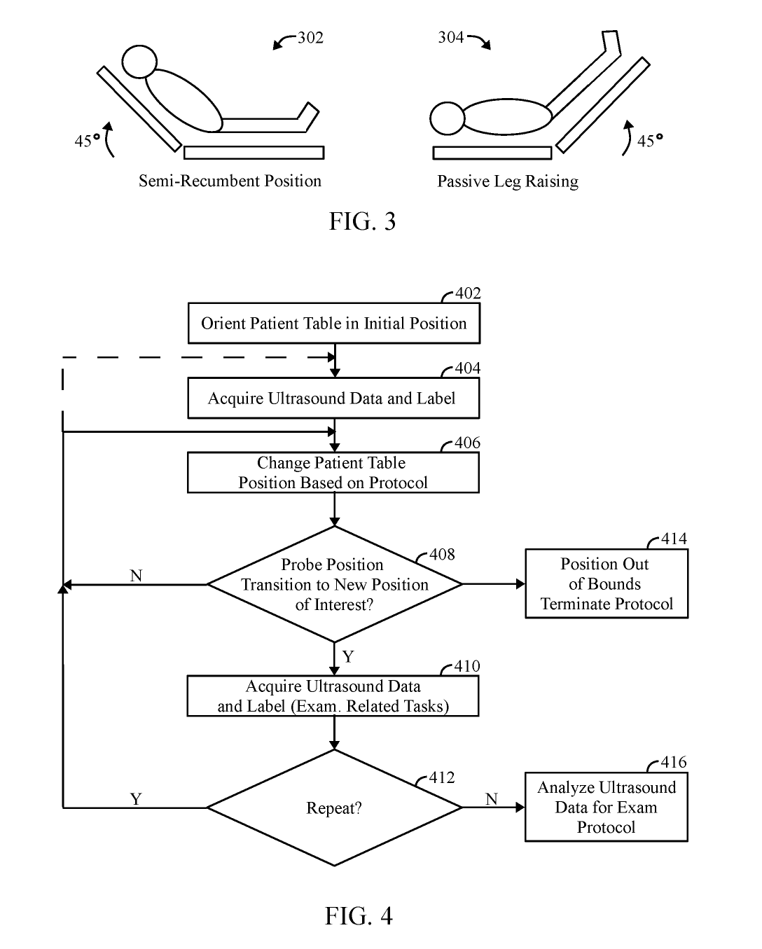

During an examination protocol, the patch probe 200 remains secured at a desired position and orientation to the patient's skin while the patient is moved between two or more positions and/or orientations as defined by the examination protocol. By way of example, when the examination protocol corresponds to a passive leg raising test, the patient is initially positioned in a semi recumbent position (corresponding to position 302 in FIG. 3). A first set of ultrasound data is collected for the carotid artery by the patch probe 200 when in the semi recumbent position 302.

FIG. 3 illustrates examples of positions, in which a patient may be positioned during a passive leg raising (PLR) examination protocol. Position 302 represents a head up semi-recombinant position in which the patient's legs are substantially horizontal while the patient's midsection and upper torso are oriented at a 45° angle with respect to horizontal. During the PLR examination protocol, the patient's upper body is lowered to horizontal and the patient's legs are passively raised until extending upward at a 45° angle with respect to horizontal (at noted at position 304). Position 302 represents a first examination related position, associated with the PLR examination protocol, while position 304 represents a second or final examination related position, associated with the PLR examination protocol. When in the final PLR position 304, the patient is rotated until the patient's midsection and upper torso are substantially horizontal while the patient's legs are elevated to be oriented at a 45° angle with respect to horizontal. As one example, the patient may be moved between positions 302 and 304 by automated motion of the bed. A second set of ultrasound data is collected automatically a predetermined time after reaching the final PLR position 304. For example, a maximum increase in stroke volume in venous return will become evident through examination of the carotid artery at approximately 30-90 seconds after the patient is moved to the final PLR position 304. As explained herein, the second set of ultrasound data is collected automatically, thereby avoiding a risk that an operation may collect the second set of data at the wrong point in time. Accordingly, the PLR examination protocol is able to compare the first and second sets of ultrasound data collected at the accurate initial and final PLR positions 302, 304. As one example, the first and second sets of ultrasound data may be analyzed to determine whether a 10% increase in stroke volume (e.g. cardiac output) is exhibited in the final PLR position 304, with respect to the initial PLR position 302.

A patient may be positioned and oriented in additional and/or alternative positions and orientations during a passive leg raising examination protocol. It is also recognized that the example examination protocol of FIG. 3 represents one non-limiting example of an examination protocol that may be utilized in accordance with embodiments herein. The examination protocol/procedure includes standardized procedures adopted by multiple positions, as well as individualized procedures that may be utilized by only a single physician or a handful of physicians. Embodiments herein may be utilized in various alternative examination protocols, some of which involve moving a patient between at least first and second examination related positions, while maintaining the ultrasound probe at a substantially fixed position and orientation on the patient's skin proximate a region of interest.

FIG. 4 illustrates the operations of a PLR examination protocol implemented in accordance with embodiments herein. The operations of FIG. 4 may be performed by one or more processors of the ultrasound imaging system 100, alone or in combination with actions by an operator. At 402, the patient is oriented in an initial position. For example, one or more processors may automatically orient the patient table in an initial PLR position (e.g., 302 in FIG. 3). Additionally or alternatively, a clinician or operator may manually orient the patient table in the initial PLR position. At 404, the one or more processors of the imaging system acquire and save, in memory, at least a first set of ultrasound data while the patient is in the initial/first PLR position. The processors also save, associated with the first set of ultrasound data, a label indicating that the ultrasound data was acquired while the patient was in the initial PLR position.

At 406, the patient and patient table position are changed based on the examination protocol. For example, the one or more processors may direct the patient table to move automatically to a next position (e.g., an intermediate PLR position, a final PLR position). Additionally or alternatively, the clinician or operator may manually adjust the patient table and the patient to the next position. Between the first and second acquisition intervals, some aspect of the patient's anatomy is physically moved or adjusted, such as changing and orientation the patient's body, raising or lowering one or more limbs, undergoing physical exertion (e.g., a stress test) and the like.

At 408, one or more processors (e.g., the position tracking circuit 125 or processor 136 of FIG. 1) analyze probe position information provided by the position sensor within the probe. For example, when the position sensor represents a gyroscope or accelerometer, the probe position information may indicate relative movement in all or a portion of the six degrees of freedom, such as speed (e.g., forward, backward, up, down, left, right, velocity, acceleration) and direction (e.g., pitch, roll, yaw). The processors of the position tracking circuit record the relative movement over time to estimate an amount and direction of movement experienced by the probe. When the probe position information indicates that the probe is moving in a particular direction and at a particular velocity, the processors track the direction and velocity over time. Based on elapsed time, the processors estimate the distance and direction that the probe (and ROI) has moved. The processors determine when the distance and direction corresponding to a transition from one examination defined patient position (e.g., based on the examination protocol) to a next examination defined patient position (e.g., based on the examination protocol). The elapsed time varies, to reach a next examination defined patient position, based upon acceleration and velocity, among other things. With reference to FIG. 3, the position tracking circuit may utilize the probe position information to track movement of the probe along an arcuate path from the initial PLR position 302 (corresponding to the semi recumbent position), to the final PLR position 304 (corresponding to the passive leg raising position).

Returning to FIG. 4, at 408, the probe position information is analyzed to determine whether the probe has moved/transitioned to a new examination patient position of interest. By way of example, at 408, the processors may identify the point of transition to the new examination patient position of interest based on distance/direction thresholds, time of movement thresholds and the like. In connection with the example of FIG. 3, the processors analyze the probe position information to correctly differentiate between raised and lowered states of a neck or other anatomy to which the probe is attached. Automating identification of the transition point removes user error and a need for user interaction with the system.

Additionally or alternatively, the thresholds may be defined relative to a reference coordinate system. For example, the processors of the position tracking circuit 125 may assign particular coordinates to the initial PLR position and the final PLR position, relative to a reference coordinate system. For example, the reference coordinate system may be defined with respect to a reference point on the patient table (e.g., proximate to a point at which the patient's neck or head is placed on the patient table when in the initial PLR position). Optionally, the reference coordinate system may be defined with respect to a reference point at another location on or proximate to the patient table, on or proximate to the imaging system, on or proximate to the position tracking circuit, or at another location within the examination room.

At 408, when the one or more processors determine that the probe position has not transitioned to a new position of interest, flow returns to either 406 or 404. Flow may return to 404 when it is desirable to obtain additional ultrasound data at the current patient/probe position. For example, more than one set of ultrasound data may be obtained at the initial PLR position. Additionally or alternatively, one or more sets of ultrasound data may be obtained at intermediate positions between the initial PLR position and a second/final PLR position. Optionally, flow may return from 408 to 406 when no additional ultrasound data is to be obtained. At 406, an additional change (automatic or manual) in the patient and probe position is made based on the examination protocol. Automating the decision at 408 avoids a risk that too little data is obtained or that the examination is rushed and final data is prematurely collected.

At 408, when the one or more processors determine that the probe position has transitioned to a new position of interest, flow advances to 410. At 410, one or more processors of the imaging system perform an examination related task. In the example of FIG. 4, the examination related task is to obtain and save in memory a new/second set of ultrasound data. The new set of ultrasound data is saved automatically with a second/final label indicating that at least a portion of the new set of ultrasound data relates to the new/second examination related position. Automating the designation and addition of the label avoids the risk of user mislabeling and removes a delay otherwise created while the user enters the label.

Optionally, at 408, the processors may determine that the probe position information indicates that the probe has moved “out of bounds,” namely outside or beyond the parameters of the examination protocol. For example, the probe position information may indicate that the patient has moved to a position/orientation, at which no meaningful ultrasound information may be acquired (e.g. the patient stands up, sits up, roles on a side and the like). When the processors determine that the probe and patient have moved to a position outside of the parameters of the examination protocol, the process of FIG. 4 moves from 408 to 414. At 414, the one or more processors terminate the examination, thereby avoiding collection of ultrasound data that is not informative relative to the examination protocol. Optionally, the identification of significant probe motion may be utilized to initiate a repositioning process in which the ultrasound system performs changes to reacquire preferred clinical information. Optionally, the operation at 414 may be omitted entirely.

At 412, the one or more processors determines whether to repeat all or a portion of the repositioning and ultrasound data acquisition. If so, returns to 404 or 406. Otherwise, the process moves to 416 where the saved sets of ultrasound data are analyzed. The passive leg raise examination protocol transiently increases venous return in patients who are preload responsive, and as such, the PLR examination protocol is a diagnostic test, not a treatment. The PLR examination protocol may be utilized at 416 to determine a level of fluid responsiveness. As one example, fluid responsiveness may be used in connection with identifying patients who are on and ascending portion of a “Starling Curve” and will experience an increase in stroke volume in response to fluid administration.

For a PLR examination protocol, the initial and final sets of ultrasound data may be compared automatically to identify characteristics within the region of interest, such as stroke volume, pulse pressure, heart rate, cardiac index and the like. In accordance with aspects herein, embodiments are provided that detect an increase or decrease in the characteristic within the region of interest, with a certain sensitivity and specificity. As non-limiting examples, a comparison of initial and final sets of ultrasound data may identify a 9% increase in stroke volume, with an 86% sensitivity and 90% specificity. As another non-limiting example, the comparison of initial and final sets of ultrasound data may identify a 10% increase in pulse pressure, with a 79% sensitivity and 85% specificity.

It is recognized that the particular orientations illustrated in FIG. 3 and described in connection with the operations of FIG. 4 are non-limiting examples.

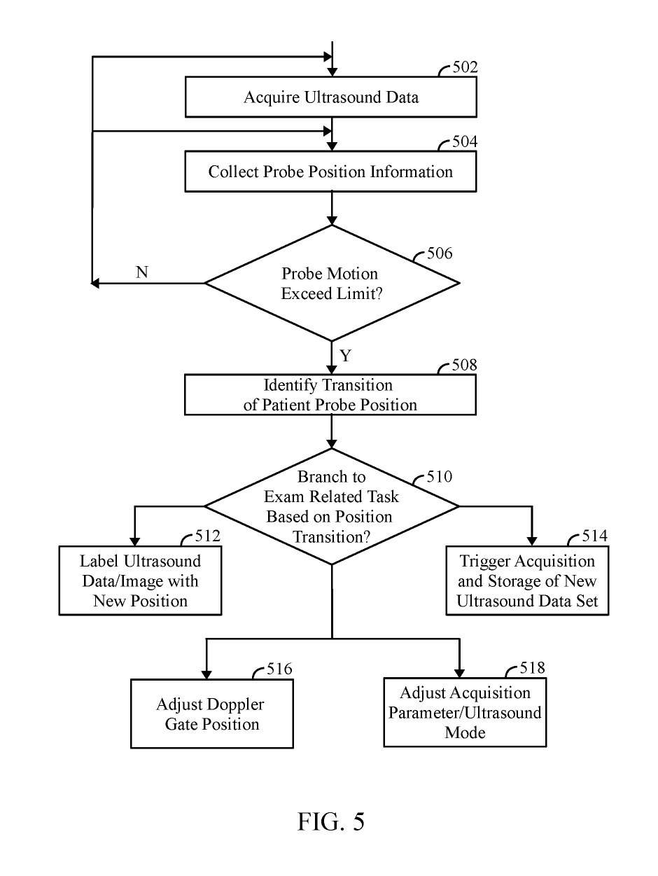

FIG. 5 illustrates a process carried out in accordance with embodiments herein for operating an ultrasound system according to an examination protocol. An examination protocol is obtained that is to be utilized while an ultrasound probe is maintained against a patient region of interest as the patient is moved between multiple examination related positions in connection with the examination protocol. At 502, a first set of ultrasound data is obtained and stored. Optionally, the first set of ultrasound data may be stored with a label designating the examination related position at which the ultrasound data was acquired. At 504, the one or more processors collect probe position information as explained herein. At 506, the one or more processors determine whether probe motion has exceeded a limit, such as a repositioning or reorientation limit. For example, the determination at 506 may include identifying when the probe moves by at least a predetermined distance from an initial position and/or moves from an initial orientation by a predetermined amount. Additionally or alternatively, the determination at 506 may be based on a change in coordinate positions, relative to a three-dimensional reference coordinate system, between an initial position and a current position. When the probe position remains within a limit of an initial position or orientation, flow returns to 502 and/or 504. At 502, additional ultrasound data is acquired. At 504, probe position information is collected. At 506, when the probe position and/or orientation exceeds the limit, flow advances to 508.

At 508, the one or more processors identify that the probe and patient have transitioned from a prior examination related position to a new examination related position. The new examination position is identified at 508. At 510, the one or more processors determine what type of examination related task is to be performed based on the transition in the examination related position. Various types of examination related task may be performed, based upon the particular examination protocol, as well as the nature of the change/transition in the probe position. For example, flow may branch to 512, where the one or more processors obtain a new label for a current set of ultrasound data and/or images. The label indicates the current examination related position. The new label may be stored in memory with the corresponding set of ultrasound data.

Additionally, at 510, flow may branch to 514, where the examination related tasks performed by the processors include triggering acquisition and storage of the ultrasound data in connection with one or more PMB transitions during at least one of the examination or monitoring protocols. The operations at 512 and 514 may be performed in combination or separately. For example, in connection with the PLR examination protocol, the one or more processors automatically labels the images with the state of the patient.

Additionally, at 510, flow may branch to 516, where the examination related tasks performed by the processors include identifying a new Doppler gate position and adjusting the Doppler gate position based thereon. An example embodiment is described herein in connection with identifying and adjusting the Doppler gate position. Additionally, at 510, flow may branch to 518, where the one or more processors of the imaging system adjust one or more acquisition parameters and/or an ultrasound mode of operation for the imaging system. Non-limiting examples of acquisition parameters that may be adjusted include gain, depth of focus, transmit and receive apertures, frequency, changes between multiline acquisition, multiline transmit and the like. Non-limiting examples of ultrasound modes include B-mode, Doppler mode, A-mode, M-mode, spectral Doppler, strain, strain rate, and the like.

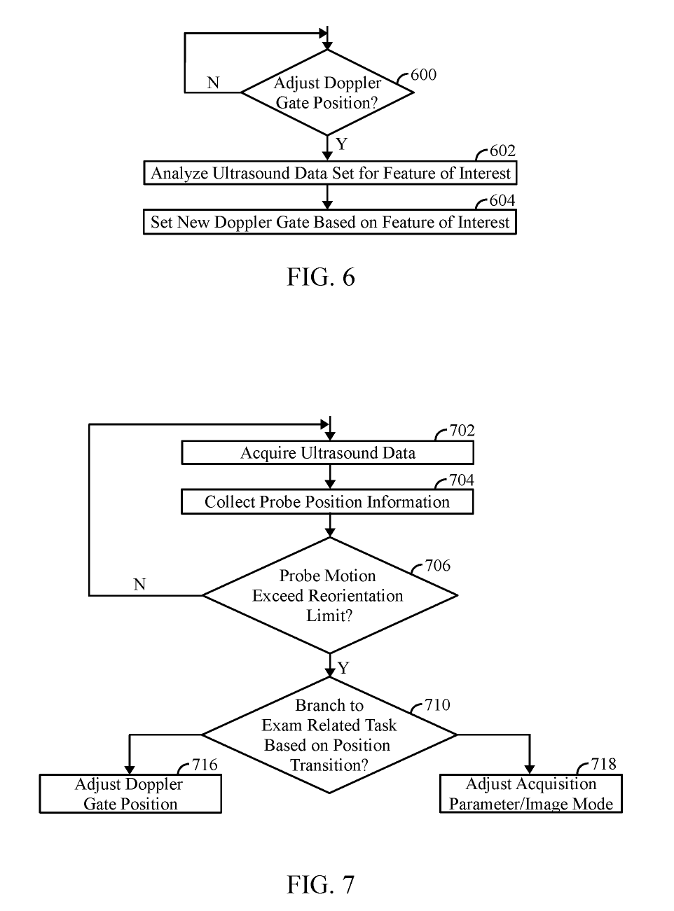

FIG. 6 illustrates a process carried out in accordance with embodiments herein for adjusting a Doppler gate position. At 600, the one or more processors determine whether a Doppler gate position should be adjusted. The determination at 600 may be based upon the degree or amount of motion detected from the probe position information. When insufficient probe position change occurs, the Doppler gate position remains unchanged. Additionally or alternatively, the decision at 600 may be based upon the content of the ultrasound data received. For example, anatomical structures within the ultrasound data may be analyzed and when the positions of the anatomical structure shift with respect to a reference point within the ultrasound image, the shift may be detected as an indication that the anatomy has changed relative to the surface of the transducer, thereby warranting an adjustment of the Doppler gate position. At 602, the one or more processors analyze the ultrasound data set for one or more features of interest. The feature of interest may correspond to an anatomical feature within an ultrasound image.

At 604, the one or more processors set a new Doppler gate based on the feature or features identified at 602. The position at which a Doppler gate is located (e.g., the center of an artery) may be identified, by the processors, based on information collected from candidate gate areas (e.g., an area that exhibit a strongest Doppler signal, fastest flow velocity and the like). Additionally or alternatively, the Doppler gate area may be designated as a location where the artery has a largest diameter. For example, when the feature of interest corresponds to the position of an anatomical feature within an ultrasound image, the new Doppler gate may be set at a position within the region of interest relative to the anatomical feature. For example, the anatomical feature may correspond to the wall of the carotid artery. The analysis at 602 may determine that the wall of the carotid artery has shifted within the field of view of the transducer by more than a predetermined limit. For example, the analysis at 602 may determine that the carotid artery wall is now proximate a center of the field of view, or not in the field of view at all. Based on the location of the carotid artery wall, a new Doppler gate may be set. Optionally, opposite side walls of the carotid artery may be identified and a central point there between selected as the position for the new Doppler gate.

Additionally or alternatively, the feature of interest may correspond to a non-imaging feature of the ultrasound data set, such as a signal-to-noise ratio, a morphology or amplitude of the Doppler signal content within the ultrasound imaging data and the like. When the feature of interest is a non-imaging feature, the analysis at 602 may identify one or more candidate positions within the field of view as potential new Doppler Gates. For example, the analysis at 602 may identify an amplitude or phase shift of Doppler signals at various positions within the field of view as candidate Doppler gates. The candidate Doppler gate exhibiting a desired characteristic (e.g., the greatest magnitude in the Doppler signal or phase shift) may be designated as the new Doppler gate position. It is recognized that alternative or additional processes may be implemented to identify a Doppler gate position.

FIG. 7 illustrates a process carried out in accordance with embodiments herein for operating an ultrasound system according to a monitoring protocol. A monitoring protocol occurs when an ultrasound probe is maintained against a patient region of interest as the patient undergoes long-term or short-term continuous or intermittent monitoring. During the monitoring protocol, a physician, clinician or ultrasound operator may or may not be present in a room. During a monitoring protocol, healthcare personnel may periodically visit and check up on the patient, but do not necessarily directly interact with the ultrasound system. For example, after a procedure, a patient may be located in a recovery room, without continuous supervision by hospital personnel.

At 702, the one or more processors acquire ultrasound data while the patient is being monitored. For example, while the patient is in the recovery room, in accordance with embodiments herein, the ultrasound system may be set to monitor one or more characteristics of the patient, such as heart rate, blood pressure, stroke volume and the like. Additionally or alternatively, ultrasound systems may be utilized to monitor non-cardiac physiologic conditions of the patient, such as to monitor the lungs, internal organs and the like. At 702, one or more sets of ultrasound data are obtained and stored. Optionally, the ultrasound data may be stored with a label designating the monitoring time, position and the like.

At 704, the one or more processors collect probe position information as explained herein. At 706, the one or more processors determine whether probe motion has exceeded a limit, such as a repositioning or reorientation limit. For example, the determination at 706 may include identifying when the probe moves by at least a predetermined distance from an initial position and/or moves from an initial orientation by a predetermined orientation amount. Additionally or alternatively, the determination at 706 may be based on a change in coordinate positions, relative to a three-dimensional reference coordinate system, between an initial position and a current position. When the probe position remains within a limit of an initial position or orientation, flow returns to 702. At 702, additional ultrasound data is acquired.

At 706, when the probe position and/or orientation exceeds the limit, flow advances to 710. At 710, the one or more processors determine what type of examination related task is to be performed based on the transition in the monitor related position. Various types of examination related tasks may be performed, based upon the particular monitor protocol, as well as the nature of the change/transition in the probe position. For example, flow may branch to 716, where the examination related tasks performed by the processors include identifying a new Doppler gate position and adjusting the Doppler gate position based thereon. An example embodiment is described herein in connection with identifying and adjusting the Doppler gate position. Additionally, at 710, flow may branch to 718, where the one or more processors of the imaging system adjust one or more acquisition parameters and/or an imaging mode of operation for the imaging system. Non-limiting examples of acquisition parameters that may be adjusted may include gain, depth of focus, transmit and receive apertures, frequency, changes between multiline acquisition, multiline transmit and the like. Optionally, the examination related task may be to trigger an alarm or warning when a patient moves more than a limit or in a particular direction or orientation.

In accordance with the operations of FIG. 7, embodiments herein support ultrasound-based continuous and intermittent monitoring of a patient without continuous direct supervision by hospital personnel. In addition, in the event a probe is shifted on the patient's skin and/or the patient moves within a bad (e.g., sits up, rolls over), embodiments herein recalibrate and correct for such changes, such as by identifying a new Doppler gate position (without user intervention), issue warnings, among other things.

In accordance with aspects described herein, the methods and systems are reversible, noninvasive and may be performed without the need for multiple operators during an examination protocol. Further, the methods and systems herein solve the need to maintain a Doppler gate at a specific location relative to an anatomy, such as at the center of the artery or otherwise. The methods and systems automatically initiate a repositioning process for the Doppler gate when probe motion is detected. Automated repositioning of the Doppler gate also affords more accurate measurements. When automated labeling is utilized, embodiments herein reduce risk of on labeling or mislabeling images that may subsequently cause error.

Embodiments herein provide methods and systems for automated ultrasound probe position registration and calculation of the position of pixels within an ultrasound image in dynamic reference to select anatomical references. The select information is stored on demand. Embodiments herein further enable, during real-time ultrasound scanning, continuous ultrasound probe position and orientation display, where the display of the probe position and orientation may be properly stored in the system's memory automatically or based on user commands. The methods and systems described herein automatically label different images associated with a common organ in connection with dynamic change in the orientation of the common organ at different points during an examination protocol. In accordance with embodiments herein, the methods and systems, based on detection of probe position change, trigger next steps in a protocol, trigger the storage of new ultrasound data, trigger changes in acquisition parameters and the like.

Embodiments herein eliminating need to manually label the steps within an examination protocol such as a PLR test. By securing a probe to a region of interest on a patient and affording an automated process for monitoring the probe position relative to an examination protocol, embodiments herein allow a single operator to perform an examination protocol. While an operator may continue to interact with the patient to facilitate repositioning and reorienting of the patient's legs, chest and the like, the operator need not step away from the patient or otherwise reach out to the ultrasound system to manually enter labels associated with steps of the examination procedure. In addition to or instead of automatic labeling, embodiments herein utilize detection of probe position changes a trigger for next steps in an acquisition process related to an examination protocol. For example, the detection of a next examination related position may trigger acquisition and storage of ultrasound data acquired during the prior or next step of the examination protocol. Further, when acquisition parameters are to be changed between the steps within a protocol, detection of the change in per position may be utilized as a basis to trigger such changes in the acquisition parameters. Further, as noted above, excessive probe motion may be utilized to initiate a repositioning process.

Embodiments herein utilize changes in probe position to initiate repositioning of a Doppler gate, such as to re-center a Doppler gate relative to an artery after detecting probe motion. The Doppler gate may be shifted in connection with electronic array type probes that move the gate position within the imaging area field of view.

The foregoing embodiments are described generally in connection with imaging probes. However, it is recognized that non-imaging probes may be utilized in connection here with. For example, a Doppler type probe may be utilized. One nonlimiting example of a non-imaging probe may utilize a transducer that is physically moved along a range of motion by an electric motor. The position at which a Doppler gate is to be located (e.g., the center of an artery) may be identified based on candidate gate areas that exhibit a strongest Doppler signal, fastest flow velocity or at a location where the artery has a largest diameter.

### Closing Statements

Various embodiments may be implemented in hardware, software or a combination thereof. The various embodiments and/or components, for example, the modules, or components and controllers therein, also may be implemented as part of one or more computers or processors. The computer or processor may include a computing device, an input device, a display unit and an interface, for example, for accessing the Internet. The computer or processor may include a microprocessor. The microprocessor may be connected to a communication bus. The computer or processor may also include a memory. The memory may include Random Access Memory (RAM) and Read Only Memory (ROM). The computer or processor further may include a storage device, which may be a hard disk drive or a removable storage drive such as a solid-position drive, optical disk drive, and the like. The storage device may also be other similar means for loading computer programs or other instructions into the computer or processor.

As used herein, the term “computer,” “subsystem,” “circuit,” “controller circuit,” or “module” may include any processor-based or microprocessor-based system including systems using microcontrollers, reduced instruction set computers (RISC), ASICs, logic circuits, and any other circuit or processor capable of executing the functions described herein. The above examples are exemplary only, and are thus not intended to limit in any way the definition and/or meaning of the term “computer,” “subsystem,” “circuit,” “controller circuit,” or “module”.

The computer or processor executes a set of instructions that are stored in one or more storage elements, in order to process input data. The storage elements may also store data or other information as desired or needed. The storage element may be in the form of an information source or a physical memory element within a processing machine.

The set of instructions may include various commands that instruct the computer or processor as a processing machine to perform specific operations such as the methods and processes of the various embodiments. The set of instructions may be in the form of a software program. The software may be in various forms such as system software or application software and which may be embodied as a tangible and non-transitory computer readable medium. Further, the software may be in the form of a collection of separate programs or modules, a program module within a larger program or a portion of a program module. The software also may include modular programming in the form of object-oriented programming. The processing of input data by the processing machine may be in response to operator commands, or in response to results of previous processing, or in response to a request made by another processing machine.

As used herein, a structure, limitation, or element that is “configured to” perform a task or operation is particularly structurally formed, constructed, or adapted in a manner corresponding to the task or operation. For purposes of clarity and the avoidance of doubt, an object that is merely capable of being modified to perform the task or operation is not “configured to” perform the task or operation as used herein. Instead, the use of “configured to” as used herein denotes structural adaptations or characteristics, and denotes structural requirements of any structure, limitation, or element that is described as being “configured to” perform the task or operation. For example, a controller circuit, processor, or computer that is “configured to” perform a task or operation may be understood as being particularly structured to perform the task or operation (e.g., having one or more programs or instructions stored thereon or used in conjunction therewith tailored or intended to perform the task or operation, and/or having an arrangement of processing circuitry tailored or intended to perform the task or operation). For the purposes of clarity and the avoidance of doubt, a general purpose computer (which may become “configured to” perform the task or operation if appropriately programmed) is not “configured to” perform a task or operation unless or until specifically programmed or structurally modified to perform the task or operation.

As used herein, the terms “software” and “firmware” are interchangeable, and include any computer program stored in memory for execution by a computer, including RAM memory, ROM memory, EPROM memory, EEPROM memory, and non-volatile RAM (NVRAM) memory. The above memory types are exemplary only, and are thus not limiting as to the types of memory usable for storage of a computer program.

It is to be understood that the above description is intended to be illustrative, and not restrictive. For example, the above-described embodiments (and/or aspects thereof) may be used in combination with each other. In addition, many modifications may be made to adapt a particular situation or material to the teachings of the various embodiments without departing from their scope. While the dimensions and types of materials described herein are intended to define the parameters of the various embodiments, they are by no means limiting and are merely exemplary. Many other embodiments will be apparent to those of skill in the art upon reviewing the above description. The scope of the various embodiments should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, the terms “first,” “second,” and “third,” etc. are used merely as labels, and are not intended to impose numerical requirements on their objects. Further, the limitations of the following claims are not written in means-plus-function format and are not intended to be interpreted based on 35 U.S.C. § 112(f) unless and until such claim limitations expressly use the phrase “means for” followed by a positionment of function void of further structure.

This written description uses examples to disclose the various embodiments, including the best mode, and also to enable any person skilled in the art to practice the various embodiments, including making and using any devices or systems and performing any incorporated methods. The patentable scope of the various embodiments is defined by the claims, and may include other examples that occur to those skilled in the art. Such other examples are intended to be within the scope of the claims if the examples have structural elements that do not differ from the literal language of the claims, or the examples include equivalent structural elements with insubstantial differences from the literal language of the claims.

What is claimed is:

1. A method for operating an ultrasound system, the method comprising:
acquiring ultrasound data with the ultrasound probe according to at least one of:
i) an examination protocol while maintaining an ultrasound probe against a patient region of interest as a patient is moved between first and second examination related positions in connection with the examination protocol; or
ii) a monitoring protocol during which an ultrasound probe is maintained against a patient region of interest;
collecting probe position information;
based on the probe position information, identifying a patient movement based (PMB) transition of the probe; and
directing the ultrasound system to automatically perform an examination related task in connection with identifying the PMB transition.

2. The method of claim 1, wherein the ultrasound data is acquired in connection with an examination protocol and the PMB transition is identified in connection with the first and second examination related positions, the examination related task includes automatically labeling a portion of the ultrasound data with a label related to at least the second examination related position.

3. The method of claim 2, further comprising forming images from the ultrasound data, wherein the examination related task includes automatically labeling a select image from the images with a label indicating whether the select image was acquired when the patient was in the first or second examination related positions, or another examination related position.

4. The method of claim 1, wherein the ultrasound data is acquired in connection with an examination protocol and the PMB transition is identified in connection with the first and second examination related positions, the ultrasound data comprises first and second ultrasound data sets that are acquired in connection with the first and second examination related positions, respectively, the examination related task including automatically labeling the second ultrasound data set with a label indicating that the second ultrasound data set was acquired while the patient was in the second examination related position.

5. The method of claim 1, wherein the examination related task includes automatically triggering acquisition and storage of the ultrasound data in connection with one or more PMB transitions during at least one of the examination or monitoring protocols.

6. The method of claim 1, wherein the examination related task includes automatically adjusting a position of a Doppler gate used in connection with acquiring at least a portion of the ultrasound data.

7. The method of claim 1, wherein the ultrasound data is acquired in connection with an examination protocol and the PMB transition is identified in connection with the first and second examination related positions, the examination related task includes automatically adjusting one or more i) an acquisition parameter or ii) an imaging mode, related to acquiring the ultrasound data, in connection with the second examination related position.

8. The method of claim 1, further comprising forming images from the ultrasound data, wherein the examination related task includes automatically labeling a select image from the images with a label indicating that the select image was acquired when the patient was in an examination related position.

9. The method of claim 1, wherein the probe position information is collected from at least one of i) a position sensor provided with the probe or ii) an analysis of the ultrasound data.

10. The method of claim 1, wherein the identifying operation comprises comparing a position limit to the probe position information as the probe position information is updated to indicate a current position of the probe as the patient is moved from a first examination related position to a second examination related position, the position limit defined relative to a reference position corresponding to a probe position when the patient is in the first examination related position.

11. An ultrasound system, comprising:
an ultrasound probe configured to be maintained against a patient proximate a region of interest, the ultrasound probe configured to acquire ultrasound data during at least one of;
i) an examination protocol while maintaining an ultrasound probe against a patient region of interest as a patient is moved between first and second examination related positions in connection with the examination protocol; or
ii) a monitoring protocol during which an ultrasound probe is maintained against a patient region of interest;
a position sensor provided with the ultrasound probe, the ultrasound sensor to collect probe position information;
memory to store the ultrasound data and to store instructions;
a processor that, when executing the instructions, is configured to:
identify a patient motion based (PMB) transition of the patient in connection with the first and second examination related positions based on the probe position information; and
direct the ultrasound system to automatically perform an examination related task in connection with identifying the PMB transition.

12. The system of claim 11, further comprising a probe mounting element to secure the probe to the patient.

13. The system of claim 11, wherein the ultrasound data is acquired in connection with an examination protocol and the PMB transition is identified in connection with the first and second examination related positions, wherein the examination related task includes automatically labeling a portion of the ultrasound data with a label related to the second examination related position.

14. The system of claim 11, wherein the memory stores ultrasound data comprises first and second ultrasound data sets that are acquired in connection with first and second examination related positions, respectively, the examination related task including automatically labeling the second ultrasound data set with a label indicating that the second ultrasound data set was acquired while the patient was in the second examination related position.

15. The system of claim 11, wherein the processor, when executing the instructions, is configured to form images from the ultrasound data, wherein the examination related task includes automatically labeling a select image from the images with a label indicating that the select image was acquired when the patient was in an examination related position.

16. The system of claim 11, wherein the processor is configured to automatically trigger, as the examination related task, acquisition and storage of a second ultrasound data set related to a second examination related position.

17. The system of claim 11, wherein the processor is configured to automatically adjust, as the examination related task, a position of a Doppler gate used in connection with acquiring at least a portion of the ultrasound data.

18. The system of claim 11, wherein the ultrasound data is acquired in connection with an examination protocol and the PMB transition is identified in connection with the first and second examination related positions, wherein the processor is further configured to form images from the ultrasound data and to automatically label a select image from the images with a label indicating whether the select image was acquired when the patient was in the first or second examination related positions, or another examination related position.

19. An ultrasound system, comprising:
an ultrasound probe configured to acquire ultrasound data;
a probe mounting element to secure the probe at a tissue attachment location on a patient;
a position sensor provided with the ultrasound probe, the ultrasound sensor to collect probe position information;
memory to store the ultrasound data and to store instructions;
a processor that, when executing the instructions, is configured to:
identify a patient motion based (PMB) transition of the probe with respect to the tissue attachment location; and
direct the ultrasound system to automatically perform an examination related task in connection with identifying the PMB transition.

20. The system of claim 19, wherein the processor is further configured to detect non-physician directed motion of the probe relative to the tissue attachment location.

21. The system of claim 20, wherein the processor is further configured to automatically adjust, as the examination related task, a position of a Doppler gate used in connection with acquiring at least a portion of the ultrasound data in response to detection of the non-physician directed motion.
